SEQUENCE LISTING (126 sequences)

SEQ ID NO: 1 - protein, 6 aa, Human immunodeficiency virus
ELDKWA

SEQ ID NO: 2 - protein, 4 aa, Human immunodeficiency virus
LDKW

SEQ ID NO: 3 - protein, 6 aa, Artificial
  epitope recognised by antibodies
ALDKWA

SEQ ID NO: 4 - protein, 6 aa, Artificial
  epitope recognised by antibodies
ELNKWA

SEQ ID NO: 5 - protein, 6 aa, Artificial
  epitope recognised by antibodies
ELDKWA

SEQ ID NO: 6 - protein, 6 aa, Artificial
  epitope recognised by antibodies
ALDTWA

SEQ ID NO: 7 - protein, 6 aa, Artificial
  epitope recognised by antibodies
QLDKWA

SEQ ID NO: 8 - protein, 6 aa, Artificial
  epitope recognised by antibodies
ELDTWA

SEQ ID NO: 9 - protein, 6 aa, Artificial
  epitope recognised by antibodies
GLDKWA

SEQ ID NO: 10 - protein, 6 aa, Artificial
  epitope recognised by antibodies
KLDEWA

SEQ ID NO: 11 - protein, 6 aa, Artificial
  epitope recognised by antibodies
ELDRWA

SEQ ID NO: 12 - protein, 6 aa, Artificial
  epitope recognised by antibodies
NWFDIT

SEQ ID NO: 13 - protein, 6 aa, Artificial
  epitope recognised by antibodies
TWFDIS

SEQ ID NO: 14 - protein, 6 aa, Artificial
  epitope recognised by antibodies
NWFDIS

SEQ ID NO: 15 - protein, 6 aa, Artificial
  epitope recognised by antibodies
SWFDIS

SEQ ID NO: 16 - protein, 6 aa, Artificial
  epitope recognised by antibodies
NWFDIS

SEQ ID NO: 17 - protein, 6 aa, Artificial
  epitope recognised by antibodies
NWFEIT

SEQ ID NO: 18 - protein, 6 aa, Artificial
  epitope recognised by antibodies
TWFSIS

SEQ ID NO: 19 - protein, 6 aa, Artificial
  epitope recognised by antibodies
NWFNIS

SEQ ID NO: 20 - protein, 6 aa, Artificial
  epitope recognised by antibodies
NWFGIS

SEQ ID NO: 21 - protein, 6 aa, Artificial
  epitope recognised by antibodies
NWFSIT

SEQ ID NO: 22 - protein, 6 aa, Artificial
  epitope recognised by antibodies
SWFNIS

SEQ ID NO: 23 - protein, 6 aa, Artificial
  epitope recognised by antibodies
NWFTIS

SEQ ID NO: 24 - protein, 6 aa, Artificial
  epitope recognised by antibodies
SWYDIS

SEQ ID NO: 25 - protein, 6 aa, Artificial
  epitope recognised by antibodies
NWFNIT

SEQ ID NO: 26 - protein, 6 aa, Artificial
  epitope recognised by antibodies
SWFSIT

SEQ ID NO: 27 - protein, 6 aa, Artificial
  epitope recognised by antibodies
SWFDIT

SEQ ID NO: 28 - protein, 6 aa, Artificial
  epitope recognised by antibodies
TWFDIT

SEQ ID NO: 29 - protein, 6 aa, Artificial
  epitope recognised by antibodies
SWFSIS

SEQ ID NO: 30 - protein, 6 aa, Artificial
  epitope recognised by antibodies
DWFSIT

SEQ ID NO: 31 - protein, 6 aa, Artificial
  epitope recognised by antibodies
NWFTIT

SEQ ID NO: 32 - protein, 6 aa, Artificial
  epitope recognised by antibodies
DWFDIT

SEQ ID NO: 33 - protein, 6 aa, Artificial
  epitope recognised by antibodies
NWFDIT

SEQ ID NO: 34 - protein, 6 aa, Artificial
  epitope recognised by antibodies
TWSDIT

SEQ ID NO: 35 - protein, 6 aa, Artificial
  epitope recognised by antibodies
NWFGIT

SEQ ID NO: 36 - protein, 6 aa, Artificial
  epitope recognised by antibodies
TWFDIT

SEQ ID NO: 37 - protein, 6 aa, Artificial
  epitope recognised by antibodies
TWFGIT

SEQ ID NO: 38 - protein, 6 aa, Artificial
  epitope recognised by antibodies
NWFAIS

SEQ ID NO: 39 - protein, 6 aa, Artificial
  epitope recognised by antibodies
NWFSIS

SEQ ID NO: 40 - protein, 6 aa, Artificial
  epitope recognised by antibodies
NWFDMS

SEQ ID NO: 41 - protein, 6 aa, Artificial
  epitope recognised by antibodies
SWFGIT

SEQ ID NO: 42 - protein, 6 aa, Artificial
  epitope recognised by antibodies
DWFSIS

SEQ ID NO: 43 - protein, 6 aa, Artificial
  epitope recognised by antibodies
SWFNIT

SEQ ID NO: 44 - protein, 6 aa, Artificial
  epitope recognised by antibodies
SWFEIS

SEQ ID NO: 45 - protein, 6 aa, Artificial
  epitope recognised by antibodies
NWFEIS

SEQ ID NO: 46 - protein, 6 aa, Artificial
  epitope recognised by antibodies
NWFNIA

SEQ ID NO: 47 - protein, 6 aa, Artificial
  epitope recognised by antibodies
TWFDIA

SEQ ID NO: 48 - protein, 6 aa, Artificial
  epitope recognised by antibodies
NWLDIT

SEQ ID NO: 49 - protein, 6 aa, Artificial
  epitope recognised by antibodies
NWLDIT

SEQ ID NO: 50 - protein, 6 aa, Artificial
  epitope recognised by antibodies
NWPDIT

SEQ ID NO: 51 - protein, 6 aa, Artificial
  consensus amino acid sequence
XWXXXX

SEQ ID NO: 52 - protein, 4 aa, Artificial
  consensus amino acid sequence
WFXI

SEQ ID NO: 53 - protein, 13 aa, Artificial
  peptide that binds IgG1-b12
HERSYMFSDLENR

SEQ ID NO: 54 - protein, 6 aa, Human immunodeficiency virus
LDKWAS

SEQ ID NO: 55 - protein, 8 aa, Artificial
  site B inclusion sequence
KNEQDLLA

SEQ ID NO: 56 - protein, 8 aa, Artificial
  site B inclusion sequence
KNEKELLE

SEQ ID NO: 57 - protein, 8 aa, Artificial
  site B inclusion sequence
KNEQELLA

SEQ ID NO: 58 - protein, 8 aa, Artificial
  site B inclusion sequence
KNEQDLMA

SEQ ID NO: 59 - protein, 8 aa, Artificial
  site B inclusion sequence
RNEKDLLE

SEQ ID NO: 60 - protein, 8 aa, Artificial
  site B inclusion sequence
KNELDLLA

SEQ ID NO: 61 - protein, 8 aa, Artificial
  site B inclusion sequence
KNEKDLLE

SEQ ID NO: 62 - protein, 8 aa, Artificial
  site B inclusion sequence
KNEQELLE

SEQ ID NO: 63 - protein, 8 aa, Artificial
  site B inclusion sequence
KNEQDLLV

SEQ ID NO: 64 - protein, 8 aa, Artificial
  site B inclusion sequence
QNEQDLLK

SEQ ID NO: 65 - protein, 8 aa, Artificial
  site B inclusion sequence
QNEQELLE

SEQ ID NO: 66 - protein, 8 aa, Artificial
  site B inclusion sequence
KNELELLE

SEQ ID NO: 67 - protein, 8 aa, Artificial
  site B inclusion sequence
KNELELLK

SEQ ID NO: 68 - protein, 8 aa, Artificial
  site B inclusion sequence
KNEQDLLE

SEQ ID NO: 69 - protein, 8 aa, Artificial
  site B inclusion sequence
ENEKELLE

SEQ ID NO: 70 - protein, 8 aa, Artificial
  site B inclusion sequence
RNEKELLE

SEQ ID NO: 71 - protein, 8 aa, Artificial
  site B inclusion sequence
KNEQELLG

SEQ ID NO: 72 - protein, 8 aa, Artificial
  site B inclusion sequence
ENEQELLE

SEQ ID NO: 73 - protein, 8 aa, Artificial
  site B inclusion sequence
KNEQALLE

SEQ ID NO: 74 - protein, 8 aa, Artificial
  site B inclusion sequence
KNELDLLE

SEQ ID NO: 75 - protein, 8 aa, Artificial
  site B inclusion sequence
KNQQELLQ

SEQ ID NO: 76 - protein, 8 aa, Artificial
  site B inclusion sequence
KNRQKLLK

SEQ ID NO: 77 - protein, 8 aa, Artificial
  site B inclusion sequence
KNEQGLLE

SEQ ID NO: 78 - protein, 8 aa, Artificial
  site B inclusion sequence
KNEQELLK

SEQ ID NO: 79 - protein, 8 aa, Artificial
  site B inclusion sequence
KNEQELSE

SEQ ID NO: 80 - protein, 8 aa, Artificial
  site B inclusion sequence
QNEKDLLA

SEQ ID NO: 81 - protein, 8 aa, Artificial
  site B inclusion sequence
ENEKDLLA

SEQ ID NO: 82 - protein, 8 aa, Artificial
  site B inclusion sequence
NNEKELLE

SEQ ID NO: 83 - protein, 8 aa, Artificial
  site B inclusion sequence
KNENDLLA

SEQ ID NO: 84 - protein, 8 aa, Artificial
  site B inclusion sequence
KNEEDLLA

SEQ ID NO: 85 - protein, 8 aa, Artificial
  site B inclusion sequence
NNEKDLLA

SEQ ID NO: 86 - protein, 8 aa, Artificial
  site B inclusion sequence
KNEKDLLA

SEQ ID NO: 87 - protein, 8 aa, Artificial
  site B inclusion sequence
QNEKDLLA

SEQ ID NO: 88 - protein, 8 aa, Artificial
  site B inclusion sequence
RNEKDLLA

SEQ ID NO: 89 - protein, 8 aa, Artificial
  site B inclusion sequence
KNEQDLLQ

SEQ ID NO: 90 - protein, 8 aa, Artificial
  site B inclusion sequence
KNEQELLQ

SEQ ID NO: 91 - protein, 8 aa, Artificial
  site B inclusion sequence
RNEQELLE

SEQ ID NO: 92 - protein, 8 aa, Artificial
  site B inclusion sequence
KNEQNLLA

SEQ ID NO: 93 - protein, 8 aa, Artificial
  site B inclusion sequence
QNEQELLA

SEQ ID NO: 94 - protein, 8 aa, Artificial
  site B inclusion sequence
RNEQELLA

SEQ ID NO: 95 - protein, 8 aa, Artificial
  site B inclusion sequence
QNEQDLLA

SEQ ID NO: 96 - protein, 8 aa, Artificial
  site B inclusion sequence
TNEKDLLA

SEQ ID NO: 97 - protein, 8 aa, Artificial
  site B inclusion sequence
RNEKDLLK

SEQ ID NO: 98 - protein, 8 aa, Artificial
  site B inclusion sequence
RNEKNLLE

SEQ ID NO: 99 - protein, 8 aa, Artificial
  site B inclusion sequence
KNEQEILA

SEQ ID NO: 100 - protein, 8 aa, Artificial
  site B inclusion sequence
KNEQELLS

SEQ ID NO: 101 - protein, 8 aa, Artificial
  site B inclusion sequence
KNEQDLLS

SEQ ID NO: 102 - protein, 8 aa, Artificial
  site B inclusion sequence
MNEQDLLA

SEQ ID NO: 103 - protein, 8 aa, Artificial
  site B inclusion sequence
KNKQDLLA

SEQ ID NO: 104 - protein, 8 aa, Artificial
  site B inclusion sequence
MNEQDLLQ

SEQ ID NO: 105 - protein, 8 aa, Artificial
  site B inclusion sequence
INERDLLA

SEQ ID NO: 106 - protein, 8 aa, Artificial
  site B inclusion sequence
KSEKDLLE

SEQ ID NO: 107 - protein, 8 aa, Artificial
  site B inclusion sequence
YNEKKLLE

SEQ ID NO: 108 - protein, 8 aa, Artificial
  site B inclusion sequence
TNEKALLE

SEQ ID NO: 109 - protein, 8 aa, Artificial
  site B inclusion sequence
TNEKSLLE

SEQ ID NO: 110 - protein, 5 aa, Artificial
  preceeding amino acid sequence
AEGEF

SEQ ID NO: 111 - protein, 28 aa, Artificial
  phage 28.3.1 peptide display
EWEDVEFELDRWALRSCCPVEGAWRWRG

SEQ ID NO: 112 - protein, 32 aa, Human immunodeficiency virus
AEGEFAKNEQELLELDKWASLWCCFNITNWLW

SEQ ID NO: 113 - protein, 30 aa, Human immunodeficiency virus
NQQEKNEQELLELDKWASLWNWFNITNWCC

SEQ ID NO: 114 - protein, 25 aa, Artificial
  modified IgG1-b12 epitope
AEGEFAAAAHERSYMFSDLENRCCI

SEQ ID NO: 115 - protein, 79 aa, Artificial
  consensus HIV-1 subtype B epitope
AEGEFKNEQELLELDKWASLSCCPDITNWLWYGDPAKAAFDSLQASATEYIGYAWAMVVVIVGATIGIKLFKKFTSKAS

SEQ ID NO: 116 - DNA, 83 bp, Artificial
  primer for consensus HIV-1 subtype 2 epitope
aaaaacgaacaggaactgctggaactggataaatgggcgagcctgagctgctgcccggtggagggcgcctggcgctggcgcgg

SEQ ID NO: 117 - DNA, 32 bp, Artificial
  primer for consensus HIV-1 subtype 2 epitope
gattgaattcaaaaacgaacaggaactgctgg

SEQ ID NO: 118 - DNA, 26 bp, Artificial
  primer for consensus HIV-1 subtype 2 epitope
aattggatccccgcgccagcgccagg

SEQ ID NO: 119 - DNA, 36 bp, Artificial
  colicin R domain plasmid primer
tggcgctggcgcgggcgacaggctaaagctgttcag

SEQ ID NO: 120 - DNA, 22 bp, Artificial
  colicin R domain plasmid primer
cctcgagctcttttgcagcagc

SEQ ID NO: 121 - DNA, 21 bp, Artificial
  28.3.1 pIF4 plasmid primer
tcaatgatgatgatgatgatg

SEQ ID NO: 122 - DNA, 38 bp, Artificial
  28.3.1 pIF4 plasmid primer
tgaacagctttagcctgtcgcccgcgccagcgccaggc

SEQ ID NO: 123 - protein, 116 aa, Artificial
  colicin R domain fusion peptide
AEGEFEWEDVEFELDRWALRSCCPVEGAWRWRGRQAKAVQVYNSRKSELDAANKTLADAIAEIKQFNRFAHDPMAGGHRMWQMAGLKAQRAQTDVNNKQAAFDAAAKELEHHHHHH

SEQ ID NO: 124 - protein, 27 aa, Artificial
  fibritin trimeric sequence of bacteriophage T4
GYIPEAPRDGQAYVRKDGEWVLLSTFL

SEQ ID NO: 125 - protein, 47 aa, Artificial
  modified 2F5 epitope
AEGEFKNEQELLELDKWASLSYIPEAPRDGQAYVRKDGEWVLLSTFL

SEQ ID NO: 126 - protein, 90 aa, Artificial
  2F5 epitope carrying peptide
RGRQAKAVQVYNSRKSELDLSCCPAANKTLADAIAEIKQFNRFAHDPMAGGHRMWQMAGLKAQRAQTDVNNKQAAFDAAAKELELVPRGS

(19) United States
(12) Patent Application Publication
(10) Pub. No.: US 2006/0275309 A1
(43) Pub. Date: Dec. 7, 2006

(54) Peptide oligomers for use as hiv vaccines

(76) Inventors: Steven Paul Boneham, Nottingham (GB); Jonathan Kelvin Ball, Nottingham (GB)

Correspondence Address: JAECKLE FLEISCHMANN & MUGEL, LLP, 190 Linden Oaks, ROCHESTER, NY, 14625-2812, US

(21) Appl. No.: 10/537,852

(22) PCT Filed: Dec. 12, 2003
(86) PCT No.: PCT/GB03/05436

§ 371 Date: Dec. 12, 2005

(30) Foreign Application Priority Data
Dec. 12, 2002 (GB) 0228939.5

(51) Int. Cl. A61K 39/42; C12P 21/04; C12Q 1/70; C07K 14/16; C07K 16/10; C12N 5/06

(52) U.S. Cl. 424/160.1; 530/350; 530/388.4; 435/70.21; 435/326; 435/5

(57) ABSTRACT

Partially occluded and/or multimeric presentations of peptides mimic the epitopes recognised by antibodies capable of neutralising diverse clinical isolates of the human immunodeficiency virus type 1 (HIV-1). By “partially occluded” is meant a presentation that has a three-dimensional structure (probably a barrel/cylindrical/helical shape) generated by inter-chain disulphide bridging or other means that has internally, at or near its base, the epitope that is recognised by the neutralising antibody; i.e. a partially occluded presentation is a three-dimensional presentation of one or more neutralising epitopes such that the epitope is located in a pocket or cleft. Such presentations are better at eliciting antibodies that have the neutralising phenotype, and may be used as vaccines or to produce antibodies for the prevention or treatment of HIV-1 infection.

This invention relates to presentations of peptides which mimic the epitopes recognised by antibodies capable of neutralising diverse clinical isolates of the human immunodeficiency virus type 1 (HIV-1). Such peptide presentations may be used as prophylactic or preventative vaccines or for the production of antibodies to be used for the prevention or treatment of HIV-1 infection. With almost 50 million individuals currently infected by the human immunodeficiency virus type 1 (HIV-1) and an estimated 16,000 new infections every day, a potent HIV-1 vaccine is needed to induce both cell-mediated and antibody responses in order to neutralise circulating virus and clear infected cells. Whilst considerable progress has been made towards interventions capable of eliciting cell-mediated immunity, the induction of potent neutralising antibodies remains a major challenge.

The listing or discussion of a prior-published document in this specification should not necessarily be taken as an acknowledgement that the document is part of the state of the art or is common general knowledge.

Protection mediated by an antibody is correlated to its ability to neutralise primary isolates of HIV-1, as opposed to T-cell-line-adapted laboratory strains. Antibodies neutralising primary isolates are present in only a minority of patient sera. Even the most potent sera tend to neutralise a limited number of isolates. To date, only a few highly conserved neutralising epitopes have been described, and these epitopes are defined by the human monoclonal antibodies 2F5, 4E10, Z13 and IgG₁-b12. Each of these antibodies mediates 90% neutralisation of diverse HIV-1 primary isolates at concentrations that are potentially achievable through vaccination. Significant synergy has been observed when some of these antibodies are combined. Potent in vitro neutralisation is correlated with in vivo protection against HIV-1 in various animal models. Therefore a vaccine capable of inducing an antibody response neutralising primary isolates is likely to provide protection against HIV-1 infection and/or disease.

HIV-1 contains two envelope proteins, gp41 which is membrane spanning and gp120 which binds to gp41. Both of these proteins are derived from a precursor protein called gp160. Linear epitopes for several neutralising antibodies have been located on gp41 (see for example EP 0 570 357 B1 and WO 00/61618 which relate to the linear epitope recognised by the antibody 2F5, and WO 03/022879 which relates to the linear epitope recognised by the antibody 4E10, each of which are incorporated herein by reference).

The human monoclonal antibody 2F5 recognises one of the few conserved epitopes recognised by broadly neutralising antibodies accessible on the gp41 sub-unit of the glycoprotein envelope of primary isolates of HIV-1. The epitope encompasses the sequence of six amino acids

Glu Leu Asp Lys Trp Ala

i.e. E L D K W A in one-letter IUPAC notation, with the core sequence L D K W identified as the most critical. The 2F5 epitope is well-conserved, with the sequence E L D K W A present in 72% of primary isolates analysed and the core sequence L D K W expressed by 80% of isolates (see, for example, Table 2(a) in EP 0 570 357 B1 and further below). However, not all such isolates are neutralised by the 2F5 antibody and even isolates with the E L D K W A epitope vary significantly in their sensitivity to neutralisation. The 2F5 antibody appears to have some binding requirements outside the E L D K W A motif. Despite the identification of the E L D K W A epitope as a potential immunogen, no vaccine candidate based on this sequence has proven capable of inducing neutralising antibodies against primary isolates of HIV-1.

Discontinuous regions within the gp120 subunit of the glycoprotein envelope of primary isolates of HIV-1 form the epitope recognised by the monoclonal antibody IgG₁-b12. The epitope overlaps the CD4-binding domain on the gp120 sub-unit, and IgG₁-b12-reactive peptide mimotopes isolated from random peptide display phage libraries show similarity at the amino acid level with the D loop of HIV-1 gp120.

Although the epitopes recognised by neutralising antibodies are likely to be present and exposed on candidate vaccines based on gp120/gp160/gp41 sub-units, in clinical trials none have been able to elicit broadly neutralising antibody responses. Data from over 2000 subjects participating in phase I and phase II clinical trials of highly purified forms of recombinant, monomeric Env vaccines demonstrated these products to be safe and generally capable of inducing humoral responses against HIV-1. However the sera from recipients of such vaccines generally failed to neutralise primary isolates of HIV-1 although the induced antibodies often neutralised T-cell-line-adapted laboratory strains. This is consistent with the minimal impact of recombinant env vaccines on the course of HIV-1 disease as reported in clinical and laboratory analyses of breakthrough infections.

The host immune response to the entire gp120/41/160 molecules of the glycoprotein envelope is usually focused on the more variable (strain-specific) and more accessible immunodominant epitopes on the monomeric subunits. Although native oligomeric forms of the envelope sub-units might serve as better vaccine candidates, the characteristics of the oligomer may limit its immunogenicity. Several domains of the gp120 sub-unit and a large portion of the gp41 sub-unit are inaccessible for antibody binding, especially in the trimeric form of gp120/41 present on the virion (virus particle). In addition, the outer exposed surface of gp120 is extensively glycosylated, which shields important epitopes from antibody binding.

We have now found that peptide mimics of the epitopes recognised by antibodies that neutralise primary isolates of HIV-1 are capable of high affinity and specific binding to the paratopes (ie the portions of the antibody that recognises the epitope) of these neutralising antibodies and, when presented in an appropriate conformation, are believed to be capable of inducing antibodies with similar neutralising profiles to the selecting antibodies.

The inherent difficulty in mimicking conformational rather than linear epitopes poses a significant problem that cannot be overcome using standard epitope-mapping methods such as pepscanning.

Peptides capable of binding to the selecting antibody have been identified among the peptides expressed as fusion proteins in selectively enriched random peptide display libraries. The technique employed to identify useful peptides in the present invention utilises a suitable host (phage or bacterium) which is genetically modified to display 6mer to 40mer peptides of a random nature which can be either linear or constrained in a disulphide loop. Particularly unusual peptides were identified when a 28mer library was screened. Multiple rounds of biopanning then enrich peptides able to bind specifically to the target antibody molecule. This is an iterative process whose success is linked to the extremely high combination of different sequences of amino acids within the inserted peptides presented in the random peptide display library. Peptides identified in this way can mimic conformational as well as linear epitopes and may also mimic interactions with non-proteinaceous (e.g. carbohydrate) antigens. New libraries for selection of peptides with improved binding kinetics and affinity may be created by mutagenising the peptide insert present in the selected host.

There is evidence that broadly reactive neutralising antibodies such as 2F5 and IgG₁-b12 have complementarity-determining regions (CDRs) which are composed of more than 6 to 8 amino acid residues, large compared with most other antibodies. In particular, the CDR3 of these antibodies is unusually large. This may partly explain why antibodies raised using linear or cyclic peptides exposed on the surface of an immunogen fail to neutralise primary HIV-1 isolates. Partial occlusion of the epitope within a pocket or cleft may select for antibodies with large CDRs following immunisation.

According to the present invention, there are provided partially occluded and/or multimeric presentations of peptides which are recognised by HIV-1 neutralising antibodies capable of neutralising diverse clinical isolates of HIV-1. The peptide which is presented is contained within a molecule, so the invention includes molecules which contain the partially occluded and/or multimeric presentations of the peptide as said. The peptide which is recognised by the HIV-1 neutralising antibody is typically a linear epitope recognised by the antibody which has been identified by any suitable technique, for example by peptide scanning. Suitable such peptides are discussed in more detail below. Preferably, the peptide is one which is recognised by any of the antibodies 2F5, IgG₁-b12, 4E10 or Z13. However, the molecule of the invention presents the linear epitope in such a way that when the molecule is used as an immunogen, neutralising antibodies are obtained. By “neutralising antibody” we include the meaning of an antibody which is capable of preventing HIV-1 infection of a cell whether in vivo or in vitro. For example, the neutralising capacity of sera or antibodies can be determined using the cell based assays described by Trkola et al (1999) J. Virol. 73, 8966-8974, incorporated herein by reference. In addition, animal model systems are well known in the art (for example, see Parren et al (2001) J. Virol. 75, 8340-8347). The standard JR-FL isolate of HIV-1 for use in neutralisation assays may be obtained from National Institutes of Health (NIH) AIDS Research and Reference Reagent Program USA (see www.aidsreagent.org; NIH AIDS Research and Reference Reagent Program, McKesson BioServices Corporation, 621 Lofstrand Lane, Rockville, Md. 20850, USA; see also Parren et al (1998) J. Virol. 72, 10270-10274; Fouts et al (1997) J. Virol. 71, 2779-2785), and the UK Centralised Facility for AIDS Reagents (see www.nibsc.ac.uk/catalog/aids-reagent/; NIBSC, Blanche Lane, South Mimms, Potters Bar, Herts., EN6 3QG, United Kingdom; Beddows et al (1999) J. Virol. 73, 1740-1745).

The molecule of the invention does not include HIV-1 and does not include gp41 or gp120 or gp160 of HIV-1. Preferably, the molecule of the invention has a monomeric molecular weight of from 2 kDa to 30 kD, typically between 2 kDa and 20 kDa, 2 kDa and 15 kDa, 2 kDa and 10 kDa, 5 kDa and 30 kDa, 10 kDa and 30 kDa and 15 kDa and 30 kDa. Typically, each monomer of the molecule of the invention has from 20 to 300 amino acid residues, typically from 25 or 30 or 35 or 40 or 50 or 60 or 70 or 80 or 90 or 100 to 300 amino acid residues.

Monoclonal antibody 2F5 is a human monoclonal antibody derived from a HIV-1 positive individual which potently neutralises a broad range of primary isolates. It was derived from asymptomatic HIV-1 (sub-type B) infected donors by the fusion of PBLs with CB-57 heteromyeloma cells and the subsequent selection of secreted antibodies against recombinant HIV-1 gp41 and gp160 (Buchacher et al (1994) AIDS Research & Human Retroviruses 10, 359-369). It is available from the National Institutes of Health (NIH) AIDS Research and Reference Reagent Program USA, and the UK Centralised Facility for AIDS Reagents (see above). Similarly, antibody IgG₁-b12, which is a human monoclonal antibody obtained from a human-derived Fab-phage display library, is available from these sources also. IgG₁-b12 (D. P. Burton, Scripps Research Institute, San Diego, Calif.) was originally derived from an antibody (Fab) display library constructed from the bone marrow RNA of an asymptomatic HIV-1 (sub-type B) infected individual. Affinity selection of this library against recombinant gp120_{IIIB} enriched for a Fab fragment (Burton et al (1991) Proc. Natl. Acad. Sci. USA 88, 10134-10137) with HIV neutralising activity. Fab IgG₁-b12 was subsequently converted to a whole IgG₁ molecule (Burton et al (1994) Science 266, 1024-1027) and expressed in CHO cells.

Antibodies 4E10 and Z13 are described in Zwick et al (2001) J. Virol. 75, 10892-10905, incorporated herein by reference. 4E10 was isolated from a hybridoma, whilst Z13 was selected from a Fab phage-display library derived from the bone marrow of a HIV-1 antibody positive individual. The linear epitopes recognised by 4E10 and Z13 overlap and they compete with each other for binding.

The linear epitopes, or at least the portions of polypeptide which contain them, which these antibodies bind are known. Thus, Table 2(a) of EP 0 570 357 B1 indicates that the following amino acid sequences bind antibody 2F5: ELDKWA (43), ALDKWA (5), ELNKWA (1), ELDKWD (1), ALDTWA (1), QLDKWA (1), ELDTWA (1), GLDKWA (1) and KLDEWA (1), where the number in parentheses indicates the number of incidence in the databases screened. In addition, as described in Example 1 below, the amino acid sequence ELDRWA forms part of a polypeptide chain which binds 2F5 as found by screening a random 28-mer phage display library.

Thus, in a preferred embodiment of the invention, the peptide is a peptide which binds to the antibody 2F5 and has an amino acid sequence comprising ELDKWA or a variant thereof in which one or two or three (preferably one or two) amino acids are replaced with another amino acid, for example such as those described above. It is particularly preferred if the peptide which binds the antibody 2F5 has the amino acid sequence ELDKWA or ELDRWA. However, it will be appreciated that a core region of these hexamers, such as the amino acid sequence LDKW may be sufficient as the linear epitope.

The following table gives amino acid sequences contained in regions which are believed to contain the linear epitopes for antibodies 4E10 and Z13 (see also Zwick et al (2001) J. Virol. 75, 10892-10905)

Amino acid positions

| Isolate No | 1 | 2 | 3 | 4 | 5 | 6 |
|---|---|---|---|---|---|---|
| 1 | N | W | F | D | I | T |
| 2 | T | W | F | D | I | S |
| 3 | N | W | F | D | I | S |
| 4 | S | W | F | D | I | S |
| 5 | N | W | F | D | I | S |
| 6 | N | W | F | E | I | T |
| 7 | T | W | F | S | I | S |
| 8 | N | W | F | N | I | S |
| 9 | N | W | F | G | I | S |
| 10 | N | W | F | S | I | T |
| 11 | S | W | F | N | I | S |
| 12 | N | W | F | T | I | S |
| 13 | S | W | Y | D | I | S |
| 14 | N | W | F | N | I | T |
| 15 | S | W | F | S | I | T |
| 16 | S | W | F | D | I | T |
| 17 | T | W | F | D | I | T |
| 18 | S | W | F | S | I | S |
| 19 | D | W | F | S | I | T |
| 20 | N | W | F | T | I | T |
| 21 | D | W | F | D | I | T |
| 22 | N | W | F | H | I | T |
| 23 | T | W | S | D | I | T |
| 24 | N | W | F | G | I | T |
| 25 | T | W | F | D | I | T |
| 26 | T | W | F | G | I | T |
| 27 | N | W | F | A | I | S |
| 38 | N | W | F | S | I | S |
| 39 | N | W | F | D | M | S |
| 30 | S | W | F | G | I | T |
| 31 | D | W | F | S | I | S |
| 32 | S | W | F | N | I | T |
| 33 | S | W | F | E | I | S |
| 34 | N | W | F | E | I | S |
| 35 | N | W | F | N | I | A |
| 36 | T | W | F | D | I | A |
| 37 | N | W | L | D | I | T |
| 38 | N | W | L | D | I | T |
| 39 | N | W | P | D | I | T |

Thus, in a preferred embodiment of the invention, the peptide is a peptide which binds to the antibody 4E10 or Z13 and has the amino acid sequence (N/D/T/S)W(FY/S/P)X(I/M)(S/T/A), where X can be any amino acid but is preferably D or another amino acid given at position 4 in the above table. Preferably, the peptide has or contains the amino acid sequence NWFNIT, SWFGIT; TWFGIT, NWFSIT (and other peptides described in WO 03/022879; incorporated herein by reference). More preferably, the peptide has or contains the amino acid sequence NWFNIT. It will be appreciated that a core region, such as WFXI may be sufficient as the linear epitope where X is any amino acid but preferably D or another amino acid given at position 4 in the above table. A peptide may be used in the context of the molecule of the invention to induce antibodies that recognise the same region of gp41 as that recognised by 4E10 or Z13.

In a further preferred embodiment of the invention, the peptide is a peptide which binds to the antibody IgG₁-b12 and has the amino acid sequence HERSYMFSDLENR (Zwick et al (2001) J. Virol. 75, 6692-6699 or a variant thereof in which one or two or three amino acids are replaced with another amino acid.

It will be appreciated that the peptide which is recognised by an HIV-1 neutralising antibody as said is preferably a peptide found in a variety of clinical isolates of HIV-1.

By “partially occluded” we include the meaning of a presentation that has a three-dimensional structure that has internally, at or near its base, the epitope that is recognised by the neutralising antibody; i.e. a partially occluded presentation is a three-dimensional presentation of one or more neutralising epitopes such that the epitope is located in a pocket or cleft.

Typically, the cleft or pocket may be from 10 Å to 20 Å deep. Such a presentation is better at eliciting antibodies that have the neutralising phenotype. Without being bound by any theory, the inventors suggest that occlusion of the peptide (linear epitope) that is recognised by a neutralising antibody in the molecule of the invention will favour the production or selection of antibodies with a longer than usual CDR3 and, as discussed above, this appears to be a feature of at least some HIV-1 neutralising antibodies.

The peptide (linear epitope) that is recognised by a neutralising antibody as said may be partially occluded in any way so that the peptide, when present in the molecule of the invention, is sufficiently accessible to the immune system in order to mount a response that gives rise to an HIV-1 neutralising antibody, but sufficiently inaccessible to deter the production of non-neutralising antibodies to the linear epitope.

The linear epitope may also be present in a multimer, typically a trimer, where although it may not necessarily be occluded, it is present in a configuration that mimics its presentation in the HIV-1 molecule. For example, if the linear epitope recognised by the neutralising antibody is one which is present in a molecule within HIV-1 which exists in a trimer configuration (such as gp41), the linear epitope is present in a trimer in the molecule of the invention. Thus, the molecule of the invention includes molecule which is a multimer of a polypeptide chain which polypeptide chain contains a linear epitope recognised by the HIV-1 neutralising antibody and a multimerisation portion wherein the polypeptide chain has a molecular weight no more than 30 kDa. Preferably, the molecule is a trimer.

Typically, a spacer portion is present so that the polypeptide chain contains the general structure: [spacer portion]-[linear epitope]-[multimerisation portion] and, optionally, [carrier portion]. Typically, these are arranged N- to C-terminal in the polypeptide chain.

Typically, the molecule of the invention is able to bind to an antibody that is raised to a clinical isolate of HIV-1 (such as those found in or derived from patient serum (such as 2F5)) but is not able to bind to an antibody which is raised against a linear or exposed version of the same epitope which the antibody binds in the clinical isolate of HIV-1. Thus, whether or not a linear epitope is partially occluded in a molecule, or is otherwise presented in a way similar to when found in HIV-1 (eg by virtue of being present in a multimer), may be determined by testing whether it binds to an antibody that is raised to a clinical isolate of HIV-1 and does not bind to an antibody raised against a linear or exposed epitope.

For example, in relation to a molecule containing a linear epitope recognised by 2F5, the molecule is a molecule of the invention if it binds 2F5, but does not bind to mouse sera that recognises the 2F5 epitope, but which do not neutralise primary isolates of HIV-1. Such sera are described in Coeffier et al (2000) Vaccine 19, 684-693, incorporated herein by reference.

Thus, the invention also includes a molecule comprising a portion which is a linear epitope which is recognised by the HIV-1 neutralising antibody and a spacer portion, typically an occluding portion. Typically, the molecule falls into the size ranges described above. Preferably, the molecule comprises one or more polypeptide chains, typically two or three or more polypeptide chains, most preferably the molecule comprises three polypeptide chains.

In a preferred embodiment, the molecule is a homomultimer (typically a trimer) of a polypeptide chain which contains a linear epitope recognised by the HIV-1 neutralising antibody and a spacer portion. Typically, the spacer portion is an occluding portion wherein the linear epitope is partially occluded by the occluding portion when the polypeptide chain is present in the multimer. In this embodiment, it is preferred if the polypeptide chain contains a spacer portion (typically an occluding portion), the linear epitope, a multimerisation portion and, optionally, a carrier portion. Conveniently, these portions are arranged in the given order from the N- to the C-terminus of each polypeptide chain.

Typically, the spacer portion consists of from 5 to 20 amino acid residues, preferably from 10 to 15 amino acid residues. In one embodiment, the spacer portion contains from 6 to 18 and typically eight, amino acid residues which are able or are predicted to form an alpha helix. The spacer portion is followed by the linear epitope which recognises the HIV-1 neutralising antibody. The linear epitope may be any of the amino acid sequences described above. Typically, the linear epitope is from 4 to 13 amino acid residues. Preferably, it is 5 or 6 or 7 or 8 amino acid residues.

The linear epitope is followed by the multimerisation portion which may be a cross-linking portion. Typically, this portion contains, in order, a spacer region of one or two amino acid residues such as glycine or serine followed by amino acid residues such as cysteine residues, that allow cross-linking to other monomers, followed by an amino acid residue such as proline capable of inducing a distortion in an alpha helix. Although cysteine residues are preferred since they are able to form disulphide bridges between monomers to form the multimer, other means of multimerising polypeptide chains are known to the person skilled in the art. For example, the multimerisation portion may contain a region of a known multimeric polypeptide which spontaneously causes homo multimerisation, such as trimerisation, by non-covalent means. For example, as described in the Examples, a portion of fibritin which leads to trimerisation may be used. Alternatively, in place of cysteine residues, amino acid residues or other chemical entities such as artificial amino acids may be included which are capable of being chemically cross-linked (eg sugar residues; Marcaurelle et al (1998) Tetrahedron Lett. 39, 8417-8420, incorporated herein by reference.

Optionally, the multimerisation region is followed by a carrier portion, but it is envisaged that it is not always required. The carrier portion may be any suitable carrier portion and included polypeptides of a sufficient size to enable the immune system to recognise and react against the molecule. Typically, the carrier portion, when present, may be 5 kDa to 10 kDa.

The carrier portion may be “passive” in the sense that it has no specific effect on the immune system other than to make the molecule a sufficient size to be recognised by the immune system.

The carrier portion may be “active” eg it may be a portion that enhances the immune response such as described below, for example it may include a polypeptide portion that is able to enhance a B cell or T cell response.

It is preferred if the carrier portion does not constitute a bacteriophage or portion thereof. It is preferred if the carrier portion does not contain a portion of an HIV-1 portion contiguous with a linear epitope as defined.

Thus, in one embodiment the polypeptide chain may be considered to have the structure A-B-C-D-E-F-G where A is a portion of from 0 to 5 amino acids, preferably 5 which may be any amino acids, B is a portion of from 5 to 15, preferably 8 amino acid residues which typically are capable of forming an alpha helix (and A and B together constitute the spacer portion), C is the linear epitope, D is an interrupting region of one or two amino acid residues, E is a portion capable of multimerisation such as forming a cross-link (such as two cysteine residues), F is a residue capable of inducing a distortion in an alpha helix (and D, E and F together constitute the multimerisation portion) and G is a carrier protein or is not present.

TABLE
Amino acid sequences for inclusion at site [B],
for example of the 2F5 immunogen, based upon the Los Alamos
HIV-1 sequence database (Kuiken et al (Eds) Theoretical
Biology and Biophysics Group, Los Alamos National Laboratory,
Los Alamos, NM, LA-UR 02-2877. HIV Sequence Compendium 2001.
These are based on the known sequences of gp41 immediately
upstream of the amino acid LDKWAS or equivalent sequence.

Amino acid position

| Isolate No | 1 | 2 | 3 | 4 | 5 | 6 | 7 | 8 |
|---|---|---|---|---|---|---|---|---|
| 1 | K | N | E | Q | D | L | L | A |
| 2 | K | N | E | K | E | L | L | E |
| 3 | K | N | E | Q | E | L | L | A |
| 4 | K | N | E | Q | D | L | M | A |
| 5 | R | N | E | K | D | L | L | E |
| 6 | K | N | E | L | D | L | L | A |
| 7 | K | N | E | K | D | L | L | E |
| 8 | K | N | E | Q | E | L | L | E |
| 9 | K | N | E | Q | D | L | L | V |
| 10 | Q | N | E | Q | D | L | L | K |
| 11 | Q | N | E | Q | E | L | L | E |
| 12 | K | N | E | L | E | L | L | E |
| 13 | K | N | E | L | E | L | L | K |
| 14 | K | N | E | Q | D | L | L | E |
| 15 | E | N | E | K | E | L | L | E |
| 16 | R | N | E | K | E | L | L | E |
| 17 | K | N | E | Q | E | L | L | G |
| 18 | E | N | E | Q | E | L | L | E |
| 19 | K | N | E | Q | A | L | L | E |
| 20 | K | N | E | L | D | L | L | E |
| 21 | K | N | Q | Q | E | L | L | Q |
| 22 | K | N | R | Q | K | L | L | K |
| 23 | K | N | E | Q | G | L | L | E |
| 24 | K | N | E | Q | E | L | L | K |
| 25 | K | N | E | Q | E | L | S | E |
| 26 | Q | N | E | K | D | L | L | A |
| 27 | E | N | E | K | D | L | L | A |
| 38 | N | N | E | K | E | L | L | E |
| 39 | K | N | E | N | D | L | L | A |
| 30 | K | N | E | E | D | L | L | A |
| 31 | N | N | E | K | D | L | L | A |
| 32 | K | N | E | K | D | L | L | A |
| 33 | Q | N | E | K | D | L | L | A |
| 34 | R | N | E | K | D | L | L | A |
| 35 | K | N | E | Q | D | L | L | Q |
| 36 | K | N | E | Q | E | L | L | Q |
| 37 | R | N | E | Q | E | L | L | E |
| 38 | K | N | E | Q | N | L | L | A |
| 39 | Q | N | E | Q | E | L | L | A |
| 40 | R | N | E | Q | E | L | L | A |
| 41 | Q | N | E | Q | D | L | L | A |
| 42 | T | N | E | K | D | L | L | A |
| 43 | R | N | E | K | D | L | L | K |
| 44 | R | N | E | K | N | L | L | E |
| 45 | K | N | E | Q | E | I | L | A |
| 46 | K | N | E | Q | E | L | L | S |
| 47 | K | N | E | Q | D | L | L | S |
| 48 | M | N | E | Q | D | L | L | A |
| 49 | K | N | K | Q | D | L | L | A |
| 50 | M | N | E | Q | D | L | L | Q |
| 51 | I | N | E | R | D | L | L | A |
| 52 | K | S | E | K | D | L | L | E |
| 53 | Y | N | E | K | K | L | L | E |
| 54 | T | N | E | K | A | L | L | E |
| 55 | T | N | E | K | S | L | L | E |

In a further preferred embodiment, the molecule comprises a first polypeptide chain which contains a linear epitope recognised by the HIV-1 antibody and a second different polypeptide chain joined to the first (whether covalently or non-covalently) which partially occludes the linear epitope on the first polypeptide chain. In this embodiment, preferably the first polypeptide chain contains a spacer region, the linear epitope, a multimerisation portion and, optionally, a carrier portion as described above for the homomultimers, and the second polypeptide chain comprises an occluding portion, a multimerisation portion and, optionally, a carrier portion. The occluding portion in the second polypeptide chain typically is from 5 to 20 (preferably 10 to 15) amino acid residues in length, and contains from 6 to 18, typically eight, amino acid residues which are able or predicted to form an alpha helix. The multimerisation portion may be any multimerisation portion as discussed above, and the carrier portion may be any carrier portion as discussed above. Typically, the second polypeptide may contain portions D to F as defined above flanked by an occluding portion which at least in part is able to form an alpha helix (typically N-terminal to portions D to F) and, optionally, a carrier portion (G) which if present is typically at the C-terminus of the polypeptide. Typically, portions D to F, or those that allow multimerisation strategies or cross-linking strategies described above, are inserted into a different protein, such as colicin, with alpha-helical structure at a position within the protein that would yield partial occlusion of the linear epitope via the formation of hetero-oligomers.

It will be appreciated that it is possible that the first polypeptide is one that may form homomultimers but, in the presence of the second polypeptide, forms heteromultimers (containing both the first and second polypeptides).

Although the polypeptide chains in the molecules of the invention may contain amino acid sequences contiguous with that found in HIV-1 polypeptides such as gp41, for example the linear epitope portion, it is preferred if the polypeptide chains contain no more than 15, 20 or 30 contiguous amino acids from an HIV-1 polypeptide.

This invention also refers to the use of such peptide presentations or molecules, either alone or in combination with a carrier molecule or as fusion proteins or as part of a chimerical organism, for the induction of antibodies capable of neutralising HIV-1, and to the antibodies thus obtained. This invention also covers the use of such peptide presentations or molecules as components of preventative or therapeutic vaccines against HIV-1, or such use of antibodies that arise following immunisation using these peptides.

The invention provides vaccines comprising such peptide presentations or molecules. The molecules or peptide presentations may be modified to facilitate induction of neutralising antibodies. For example, the molecule or peptide presentation may be administered with an adjuvant or coupled to a carrier molecule, or it may be expressed as a fusion protein or as a recombinant or chimerical prokaryotic or eukaryotic organism.

The peptide presentation or molecule may be used with an appropriate carrier molecule, such as tetanus toxin or keyhole limpet haemocyanin. Alternatively the molecule or peptide presentation may be cloned so that it can be expressed in the context of well-characterised fusion proteins, for example thioredoxin or phage proteins. In addition, molecules or peptide presentations can be presented in the context of immune-accessible proteins of various organisms to produce a recombinant or chimeric vaccine. The induction of mucosal as well as peripheral antibody responses may result from alterations in the route of administration or of the background organism used to produce the recombinant immunogen. Common recombinant organisms include recombinant modified vaccinia Ankara, Mycobacterium bovis BCG and Salmonella typhimurium. Immunogenicity of the peptide may also be enhanced by presenting it alongside other immune signals, e.g. cytokines, or by modifying the release of the antigen, e.g. by controlling its delivery from polymeric microspheres.

Since the molecule of the invention may be a polypeptide of one or more polypeptide chains, one or more of the polypeptide chains may be chemically synthesised using standard peptide synthetic chemistry or, more conveniently, expressed from a polynucleotide encoding the polypeptide chain. Thus, a further embodiment of the invention provides a polynucleotide encoding a polypeptide chain of the invention. The polynucleotide may be DNA or RNA but is preferably DNA. Similarly, the invention includes vectors comprising the polynucleotide such as expression vectors, and host cells which contain the polynucleotide or vector. Suitable host cells include bacteria such as E. coli, and yeast, insect and mammalian cells. The polynucleotides, vectors and host cells can readily be made using methods well known in the art of molecular biology such as those described in Sambrook & Russell (2001) “Molecular cloning, a laboratory manual”, 3^{rd} Edition, Cold Spring Harbor Laboratory Press, Cold Spring Harbor, N.Y., USA.

The molecules and trimeric or multimeric presentations of the invention are useful in medicine, particularly in vaccine production. In some circumstances the polynucleotide, particularly DNA, of the invention may be used to give rise to an immune response to the encoded polypeptide. Thus, the invention includes the polynucleotides for use in medicine.

Similarly, it is convenient if the molecules, peptide presentations and polynucleotides of the invention are included in a pharmaceutical composition in combination with a pharmaceutically acceptable carrier. The molecules and peptide presentations of the invention may conveniently be administered in combination with a compound which enhances the B cell response such as QuilA or a B cell cross linker (such as B-cell receptor antibodies as described in Vos (2000) Immunol. Rev. 176, 154-170 or with a compound that enhances a T cell response such as interleukin (IL)-2, IL-4 or IL-5, or both.

Pharmaceutically acceptable carriers are generally sterile and pyrogen free.

It will be appreciated from the foregoing that the molecule or presentation of peptides or polynucleotide may be used to induce neutralising antibodies in an immunised host organism. The immunised host organism may be any animal. In the context of treatment, the animal is preferably man. In the context of the production of antibodies, the animal is typically a laboratory animal such as a mouse or rabbit, or may be a cow or sheep.

Thus, the invention also includes a method of obtaining an HIV-1 neutralising antibody, the method comprising administering a molecule or peptide presentation or polynucleotide of the invention to an animal, allowing the animal to produce antibodies, and recovering the antibodies directly or indirectly from the animal. By “directly” from the animal, the antibodies are those typically found in the serum of the animal which has been administered the molecule, presentation of peptides or polynucleotide. By “indirectly”, we include monoclonal antibodies which are made from a hybridoma or similar cell derived by using the animal which has been administered the molecule, presentation of peptides or polynucleotide.

The invention also includes a method of obtaining an HIV-1 neutralising antibody, the method comprising selecting an antibody from an antibody display library in vitro which binds to a molecule or presentation of peptides of the invention, and synthesising an antibody containing the binding determinants of the so selected antibody.

The antibodies of the invention include whole antibodies, antibody fragments and genetically engineered antibodies. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299. By “ScFv molecules” we mean molecules wherein the V_{H} and V_{L} partner domains are linked via a flexible oligopeptide.

The invention also includes an antibody obtained by using these methods. The antibody may be useful for passive immunisation of an individual or to treat HIV-1 infection or to prevent HIV-1 infection. Thus, the invention also includes the antibody for use in medicine and a pharmaceutical composition comprising the antibody and a pharmaceutically acceptable carrier.

The molecule, peptide presentation, polynucleotide and antibodies of the invention are useful in treating or preventing HIV-1 infection in an individual, and may be used in the manufacture of a medicament for treating or preventing HIV-1 infection.

The invention also includes a method of detecting HIV-1 neutralising antibodies in a sample the method comprising contacting the sample with a molecule or trimeric or multimeric presentation of the invention and determining whether any antibodies present in the sample bind thereto.

The sample is any sample suspected of containing HIV-1 antibodies but typically is from an individual suspected of being infected with HIV-1. Typically, the sample is blood or serum.

Determining whether an antibody in the sample binds to the molecule of the invention may be done in any suitable way, for example by immobilising the molecule on a surface and determining whether any antibodies from the sample bind and are co-immobilised. Other suitable methods include ELISA-type methods and the like.

Thus the molecules and presentation of peptides of the invention can be used to distinguish between antibodies with a primary-isolate neutralising and non-neutralising phenotype. Those antibodies whose epitopes are defined by the IgG₁-b12 and 2F5, 4E10, Z13 or other antibodies with the primary isolate neutralizing phenotype react with both the multimeric and monomeric presentations of the peptides in non-reducing Western blot analysis (when the multimeric forms are linked by a group, such as a disulphide group, which is sensitive to the oxidation state). By contrast, those antibodies whose epitopes are defined by, for example, the IgG₁-b12 and 2F5, 4E10, Z13 antibodies that are not capable of neutralising primary isolates of HIV-1 will not react with the multimeric/oligomeric presentation. Thus, preferably the molecules of the invention are used under conditions which retain their integrity eg multimeric nature.

It will be appreciated that the invention includes screening methods which have made use of relative large peptides to identify features which are relevant to the binding of HIV-1 neutralising antibodies beyond the linear epitope. Thus, a further aspect of the invention provides a method of identifying a molecule which may be useful in raising a neutralising response to HIV-1 the method comprising screening a peptide display library wherein the displayed peptides are from 15 to 40 amino acids in length with an HIV-1 neutralising antibody and selecting those displayed peptides which bind to the antibody.

Typically, the peptide display library is a bacteriophage display library and, conveniently, the peptide displayed in around 20 to 40 amino acid residues, such as 28 amino acid residues.

Conveniently, it is further determined whether the displayed peptides are able to bind to an antibody raised against a linear epitope recognised by the HIV-1 neutralising antibody and selecting those displayed peptides that are not able to so bind. By linear epitope we mean one which is exposed and not occluded or presented as a multimer as in the molecules of the invention.

Preferably, the HIV-1 neutralising antibody is any of 2F5, IgG₁-b12, 4E10 and Z13 as discussed above.

In a further embodiment, the method further comprises determining whether the so-selected molecule is one which is or is able to form a multimer, for example by disulphide cross-linking. This may be achieved by determining whether or not the HIV-1 neutralising antibody is able to bind under reducing or non-reducing conditions as discussed above and in the Examples. Molecules obtained by this method are also included in the invention.

The following non-limiting Examples illustrate the invention with reference to monoclonal antibodies 2F5, 4E10, Z13 and IgG₁-b12 which have the ability to neutralise primary isolates of HIV-1.

### EXAMPLE 1

### Affinity Selection of 28Mer Phage Peptides Against Antibody 2F5

Affinity Selection

Affinity selection of the pC89/pIF4 type 8+8 phagemid random peptide display library was used in biopanning experiments to identify peptides capable of specific binding to human monoclonal antibody 2F5, which is a human IgG₁ molecule specific for the gp41 sub-units of the glycoprotein envelope of HIV-1. The antibody was derived from a donor infected with asymptomatic HIV-1 (sub-type B) by the fusion of peripheral blood mononuclear cells with CB-57 heteromyeloma cells and the subsequent selection of secreted antibodies against recombinant HIV-1 gp41 and gp160 (A. Buchacher & al (1994) “Generation of human monoclonal antibodies against HIV-1 proteins; electrofusion and Epstein-Barr transformation for peripheral blood lyphocyte immortalisation” AIDS Research and Human Retroviruses 10: 359-369).

The pC89/pIF4 phage vectors carry an ampicillin resistance marker, an fd Florigin of replication and a recombinant gene 8 (g8) expressing random peptides at position six of the mature p8 capsid protein. The displayed peptides are therefore preceded by the amino acids
Ala Glu Gly Glu Phe
   i.e. A E G E F in one-letter notation). The expression level of recombinant p8 is under the control of a pLac promoter inducible with isopropylthiogalactoside and can theoretically vary between 1 and 2700 copies per phage. The pIF4 vector is a derivative of pC89 in which the native g8 leader sequence of the M13 phage of Escherichia coli is replaced by that of pelB. When bacteria containing pC89/pIF4 phagemids are superinfected with M13K07 helper phage, the hybrid M13 phagemid particles are secreted into the culture supernatant.

Affinity selections from the pC89/pIF4 phagemid libraries were performed using published methods (F. Felici & al (1991) “Selection of antibody ligands from a large library of oligopeptides expressed on a multivalent exposition vector” Journal of Molecular Biology 222, 301-310), with modifications as described below. One major modification was the elution of phage binding to the selecting molecule through competition with an appropriate ligand to favour the isolation of biologically active peptides. Thus for antibodies to the HIV-1 envelope glycoproteins, gp160 was used for elution. An oligomeric gp160 (o-gp160_{IIIB}; Autogen Bioclear, Wiltshire, UK) was chosen for these experiments, as this may be more representative of the native form of this protein on the HIV-1 virion. Following this affinity selection, the enriched libraries were expanded in order to reduce the potential for the accidental loss of rare target-binding clones during subsequent rounds.

In brief, affinity selection of pC89 phagemid random peptide display libraries, containing random peptide inserts of 9 and 28 amino acids respectively, was performed in the wells of a maxisorp microtitre plate (Nunc, Roskilde, Denmark). Monoclonal antibody 2F5 was coated to these wells at a concentration of 10.0 to 100 μgml⁻¹ by overnight incubation in 100 μl of coating buffer (0.05 M carbonate-bicarbonate, pH 9.6) at 4° C. Wells were then blocked for 1 hour at 4° C. with 300 μl of TBS-TB (Tris-buffered saline, pH 7.6 (TBS) containing 0.1% v/v Tween 20 and 1.0% v/v bovine serum albumin (BSA)). After discarding the blocking solution, wells were washed three times with TBS-T (TBS containing 0.1% v/v Tween 20) before the addition of 1×10¹¹ phage from an equimolar mix of pC89 and pIF4 phagemid random peptide display libraries, diluted in 100 μl TBS-T. These phage were allowed to bind immobilised proteins for 1 hour at 25° C. before the unbound phage were removed through ten serial washes with TBS-T. Bound phage were eluted with 100 μl of competitor molecule (o-gp160_{IIIB}) diluted to 10 μgml⁻¹ in TBS.

Following elution, the enriched libraries were expanded by the infection of 1×10⁵ phage into 300 μl of log phase Escherichia coli ER2537. The outgrowth of these infected bacteria was then performed by the addition of 1.0 ml Luria broth (LB) containing 0.5 μgml⁻¹ ampicillin, followed by incubation at 37° C. for 30 minutes. This culture was then plated on LB agar containing 1% glucose and 100 μgml⁻¹ of ampicillin in 230 mm² bio-assay plates. Glucose was used as a metabolic repressor of the pLac promoter to reduce the effects of any biological bias associated with the expression of recombinant peptides. After overnight growth at 37° C., bacteria were collected and transferred into 5 ml LB-GA (LB containing 10% glycerol and 100 μgml⁻¹ of ampicillin) using sterile cell-scrapers (Philip Harris Ltd., UK). Bacteria were then filtered through sterile muslin to remove agar and other debris and the filter flushed through with an additional 5 ml LB-GA to collect the remaining cells. The absorbance of this culture was read at 600 nm and the concentration of recovered bacteria estimated (1.0 OD₆₀₀ₙₘ≡3.3×10⁸ bacteria ml⁻¹).

An appropriate volume of this culture was then transferred to 5 ml of LB containing 100 μgml⁻¹ ampicillin to give a final OD₆₀₀ₙₘ of 0.05 and this culture was then grown to an OD₆₀₀ₙₘ of 0.3 at 37° C. Bacteria were then super-infected by the addition of M13K07 helper phage at an MOI of 30 before growing for a further hour at 37° C. Bacteria were transferred to a sterile 250 ml baffled culture flask containing 30 ml LB supplemented with 100 μgml⁻¹ ampicillin and 50 μgml⁻¹ kanamycin and grown overnight (16 hours) at 37° C. Bacteria were cleared by centrifugation at 10 000×g for 10 minutes and phage subsequently recovered from the supernatant of this culture by the addition of one fifth volume of PEG (20% w/v polyethylene glycol-8000, 2.5 M sodium chloride) for 1 hour at 4° C. and collected by centrifugation at 15000×g for 20 minutes.

Phage pellets were then re-suspended in 1.0 ml TBS before a second precipitation with one fifth volume of PEG, for 1 hour on ice. Phage were subsequently collected by centrifugation at 10 000×g for 20 minutes (MSE Micro Centaur) and re-suspended in a final volume of 200 μl sterile TBS. An input of 1×10¹² phage was subsequently used as the input to a further two or three rounds of affinity selection. For the final round, reducing the incubation period of the selecting molecule with the enriched libraries from 1 hour to 10 minutes increased the stringency of selection.

Following the final round of affinity selection, individual members of the enriched library populations were isolated and the amino acid sequence of the displayed peptides was deduced by sequencing of the recombinant DNA encoding them.

The nomenclature used throughout this example to identify clones derived from a phagemid random peptide display library is based on a combination of the size of peptide displayed, the round of enrichment from which clone was derived, and a unique clone number. Thus clone 28.3.1 represents the first clone isolated after three rounds of affinity selection of a 28mer phagemid random peptide display library.

Following affinity selection of the pC89/pIF4 gene 8 28mer phagemid random peptide display libraries, the majority of selected clones had a primary amino acid sequence that shared homology to the 2F5-epitope. The random peptide insert for five out of twelve clones was of the single sequence of 28 amino acids represented by phage 28.3.1 and shown in the following formula

Glu Trp Glu Asp Val Glu Phe Glu Leu Asp [symbol] Trp Ala Leu
1 2 3 4 5 6 7 8 9 10 11 12 13 14
Arg Ser Cys Cys Pro Val Glu Gly Ala Trp Arg Trp Arg Gly
15 16 17 18 19 20 21 22 23 24 25 26 27 28
i.e. E W E D V E F E L D [symbol] W A L R S C C P V E G A W R W R G

one-letter notation.

(Compared with the gp41 sequence of the H×B2 strain, identical amino acids are shown in bold type and related amino acid in bold italic type. The adjacent cysteine residues are underlined.)

The sequence of 28 amino acids in this phage peptide has two striking features: (i) there are regions of homology to the sequence of the sub-unit gp41 of the HIV-1 envelope glycoprotein outside the previously described epitope; and (ii) there are adjacent cysteine residues within the peptide sequence. This phage peptide may have conformational complexity not seen in the smaller peptide inserts.

Disulphide Bridging in Phage Peptide 28.3.1

The adjacent cysteine residues in the 28mer phage peptide 28.3.1 may form inter-molecular covalently-linked complexes leading ultimately to a partially occluded presentation of the epitope, or one that better mimics the epitope as presented on the native virion. This was investigated by carrying out sodium dodecyl sulphate/polyacrylamide gel electrophoresis/western blotting of the phage peptide, with a phage peptide selected against monoclonal antibody 2G12—as negative control and in some cases a 9-mer (instead of 28mer) phage peptide selected against monoclonal antibody 2F5 as positive control. Proteins were resolved by sodium dodecyl sulphate/polyacrylamide gel electrophoresis.

Acrylamide resolving gels (8.0 to 16.0%) were cast in an Atto blot gel apparatus (Atto, Japan) assembled according to the manufacturers instructions. Resolving gel solution (10 ml) was prepared as described by E. Harlow & D. Lane in “Antibodies: a laboratory manual” (Cold Spring Harbour Laboratory, Cold Spring Harbour, N.Y. 1988) by combining the appropriate volumes of easigel (30.0% acrylamide:bisacrylamide solution (ratio 37.5:1), Anachem), 1.5 M Tris (pH 8.8), 10.0% sodium dodecyl sulphate, 10.0% ammonium persulphate, N,N,N′,N′-tetramethylethylenediamine and distilled water. Gels were poured immediately, overlaid with distilled water and allowed to polymerise. Once polymerised, the surface of the resolving gel was flushed with distilled water before overlaying with a 5.0% polyacrylamide stacking gel prepared by combining the appropriate volumes of easigel, 1.0 M Tris (pH 6.8), 10.0% sodium dodecyl sulphate, 10.0% ammonium persulphate, N,N,N′,N′-tetramethylethylenediamine and distilled water.

Aliquots containing 5×10¹⁰ phage particles were either untreated or treated with 3 μl of 0.27 M N-ethylmaleimide (Sigma) or 3 μl of 0.54 M dithiothreitol (Sigma), incubated for one hour at 4° C., then mixed with an equal volume of 2× Laemmli buffer (4% sodium dodecyl sulphate, 200 mM dithiothreitol, 120 mM Tris (pH 6.8), 0.002% bromophenol blue) and heated to 85° C. for 5 minutes (M. B. Zwick & al (2001) “Identification and characterisation of a peptide that specifically binds the human immunodeficiency virus type 1 antibody IgG₁-b12” Journal of Virology 75, 6692-6699). Aliquots of 10.0 μl were loaded into the wells of the polymerised stacking gel then resolved by electrophoresis at a constant voltage of 150 V for 1.0 to 2.0 hours in Tris-glycine buffer (25.0 mM Tris, 250 mM glycine, 0.1% sodium dodecyl sulphate). To assess the molecular weight of the proteins detected by western blot, all gels were run with either high or low range rainbow molecular weight markers (Amersham-Pharmacia) as appropriate.

For immunological detection, resolved proteins were transferred to polyvinylidene difluoride membranes that had been equilibrated by sequential incubations with 100% ethanol and transfer buffer (48 mM Tris, 39 mM glycine, 0.037% v/v sodium dodecyl sulphate, 20% v/v methanol). Transfer was performed using the Trans-Blot SD semi-dry transfer cell apparatus (Biorad) run at 25 V for 1.5 hours. Membranes were then blocked with Tris-buffered saline, pH 7.6 (TBS) containing 0.1% Tween 20, and 3% dry milk powder (TBS-TM) for 1 hour at 25° C. before overnight incubation with primary antibodies diluted to 1.0 μgml⁻¹ in TBS-T. Primary antibodies used were 2F5, a rabbit anti-phage polyclonal serum (Sigma) and mouse serum raised against the 2F5 epitope displayed in the context of Escherichia coli MalE protein (E. Coeffier & al (2000) “Antigenicity and immunogenicity of the HIV-1 gp41 epitope ELDKWA inserted into permissive sites of the MalE protein” Vaccine 19, 684-693). After washing with Tris-buffered saline, pH 7.6 (TBS) containing 0.1% Tween 20 (TBS-T), membranes were incubated with the appropriate horse radish peroxidase (HRP)-conjugated antibody diluted to 1:2000 in TBS-T for 2 hours at 25° C. Following a final series of washes, the membrane was either incubated with 3,3′-diaminobenzidine before drying briefly under a vacuum at 85° C. or incubated with electrochemiluminescent substrate (Amersham) for 1 minute before exposing to X-ray film for 1 to 30 minutes.

The rabbit-anti-phage serum (of rabbit anti-fd antibody) demonstrated the presence of phage proteins in the untreated 28.3.1 phage peptide and negative control phage peptide preparations. Probing the blotted native untreated protein samples with 2F5 antibody showed the presence of 2F5-reactive p8 fusion protein only in the 28.3.1 clone. There were several molecular weight forms of the 28.3.1 protein apparent. One migrated with an approximate molecular mass of 6.5 kDa, presumed to be the monomeric form, other bands with predicted masses greater than 14 kDa. The latter were presumed to be oligomeric forms of the p8 fusion protein. A similar pattern was observed for the protein treated with N-ethyl maleimide. This suggests that the oligomeric forms of the protein exist on the surface of the phage rather than forming after lysis of the phage particles. Following treatment with dithiothreitol only the predicted monomeric form of the 28.3.1 p8 fusion protein was detected, suggesting that the oligomeric forms were indeed formed by disulphide bridging between adjacent monomeric p8 units. No multimeric complexes of the p8 fusion peptide were seen in any other phage clones generated during the biopanning experiments nor did these clones possess adjacent cysteine residues in the peptides inserts.

Immunoassay of Phage Peptide 28.3.1

To assess the accessibility of the predicted 2F5 epitope on the monomeric and oligomeric p8 fusion protein present in clone 28.3.1, further western blots were performed using mouse sera raised against the 2F5 epitope expressed in MalE protein; these sera are known not to be able to neutralise primary isolates of HIV-1 (Coeffier et al (2000) Vaccine 19, 684-693). The mouse sera detected the monomeric presentation of the untreated native 28.3.1 p8 fusion protein and 28.3.1 p8 fusion protein treated with N-ethyl maleimide but not the oligomeric form. As expected, probing western-blotted 28.3.1 phage protein treated with dithiothreitol revealed the presence of a single band representative of monomeric p8 fusion protein.

In enzyme immunoassays the 28.3.1 clone demonstrated high and specific reactivity to the 2F5 antibody compared to an HIV-1-specific 2G12 antibody as negative control.

To perform the immunoassays, antibodies were diluted to between 1.0 and 100 μgml⁻¹ in 50 μl carbonate bicarbonate buffer and coated directly into the wells of a microtitre plate by overnight incubation at 4° C. Following blocking with TBS-TM, approximately 1×10¹¹ phage particles were added to the wells and allowed to bind for 1 to 2 hours. Bound phage were then detected by incubation with either the rabbit anti-fd antibody or a serum raised against wild-type M13 phage by immunisation of mice, both diluted to 1:1000. The binding of these antibodies was then detected by sequential incubations with an appropriate secondary antibody and substrate and the absorbance at 490 nm recorded as described. The cut-off above which phage were considered to be reactive with an antibody or protein was defined as the mean OD₄₉₀ₙₘ of reactivity to wild-type M13 plus three standard deviations. The absorbance of the 28.3.1 phage samples was consistently above the cut-off of the assay.

Conclusion and General Discussion

Taken together, the data in this example show that the 28.3.1 p8 phage fusion protein, containing the peptide sequence displayed above, exists on the surface of the phage as both a monomer and a multimer promoted by inter-p8 disulphide bonds between the cysteine residues present at positions 17 and 18. This multimeric presentation may partially occlude the 2F5 epitope from non-neutralising mouse antibodies, although the mouse serum does recognise the monomeric presentation of the epitope. By contrast the epitope is accessible to neutralising 2F5 antibody on both the monomer and the trimer.

It appears therefore that partially occluded and/or multimeric presentations of HIV-1-neutralising epitopes preferentially select for antibodies with a virus-neutralising phenotype, and that they are therefore better immunogens in terms of their potential for HIV vaccines or treatments than the linear or surface-exposed presentations of these epitopes previously described. It further appears that such a presentation may allow simultaneous contact between the antibody and the epitope present on different sub-units, or that such a presentation mirrors the presentation of the epitope on the trimeric gp41 sub-units of the glycoprotein envelope of primary isolates of HIV-1.

The three-dimensional structure (probably a barrel/cylindrical/helical shape) is formed by the three linked peptide monomers (in this case as presented on the phage major coat protein p8, but there could be a similar presentation on any carrier molecule). The depth at which the epitope is located in a pocket or cleft within the cylinder/barrel/helix may be variable, but the depth and the primary amino acid sequence are each sufficient to encourage the selection of monoclonal antibodies with complementarity-determining regions that are sufficiently long to interact with the epitope as it is present on native primary HIV-1 isolates. In the case of the epitope described in this example, the 28 amino acid sequence is preceded at the N-terminal end of the phage gene 8 protein by the amino acid sequence Ala Glu Gly Glu Phe, making the total length of the amino acid peptide upstream of the adjacent cysteine residues 21 amino acids long.

Several possible disulphide-bridged states exist in the multimeric presentation. These can be assigned according to the sub-unit the cysteine residue is located on and the relative position of the cysteine residue on the primary amino acid sequence. By way of illustration, a trimer presented in the 28.3.1 p8 phage fusion protein might for example have disulphide bridging between the cysteine at position 16 on sub-unit I (I-C₁₆) and the cysteine at position 16 on sub-unit III (III-C₁₆) together with bridging between the cysteine at position 17 on sub-unit III (III-C₁₇) and the cysteine at position 16 on sub-unit II (II-C₁₆) and together with bridging between the cysteine at position 17 on sub-unit II (II-C₁₇) and the cysteine at position 17 on sub-unit I (I-C₁₇). (The trimer might thus be represented as I-C₁₆-S-S-III-C₁₆, III-C₁₇-S-II-C₁₆, II-C₁₇-S-S-I-C₁₇.) This invention relates to all possible combinations of partially and completely disulphide-bonded multimeric forms.

The partially occluded presentation should be such as to exclude access of the whole immunoglobulin molecule or B-cell to the epitope, thereby preferentially selecting antibodies that have complementarity-determining regions that would be capable of accessing or interacting with the epitope as presented on the native virion (virus particle).

### EXAMPLE 2

### Partially Occluded Presentation of Native Epitope of Antibody 2F5

A partially occluded presentation according to the invention may also generally be used to present the native epitope as it appears on virus isolates or other linear peptide sequences shown to be reactive with a neutralising monoclonal antibody. This example shows a disulphide-bridged presentation of the HIV-1 (strain H×B2) sequence encompassing the 2F5 epitope:

Ala Glu Gly Glu Phe Ala Lys Asn Glu Gln Glu Leu
Leu Glu Leu Asp
Lys Trp Ala Ser Leu Trp Cys Cys Phe Asn Ile Thr
Asn Trp Leu Trp
carrier
i.e. A E G E F A K N E Q E L L E L D K W A S L W C C F N I T N W L W-carrier

in one-letter notation. The amino acid residues forming an additional N-terminal flanking sequence to help maintain the depth of the pocket/cleft within the trimer are in bold type.

### EXAMPLE 3

### Presentation of 4E10/Z13 Epitope and a Combination of the 2F5 Epitope and 4E10/Z13 Epitope

Whilst the primary amino acid sequences of the epitopes or mimotopes recognised by monoclonal antibodies 2F5 and IgG₁-b12 have previously been described, there is novelty in the method of presentation of these epitopes. Generally, this invention also covers such a presentation, as a trimeric or multimeric and/or partially occluded epitope, of epitopes recognised by other HIV-1 neutralising antibodies such as 4E10 and Z13 capable of neutralising diverse clinical isolates of HIV-1.

The epitopes defined and recognised by the Z13 and 4E10 monoclonal antibodies are similarly presented in a multimeric and/or partially occluded presentation, either alone or in combination with each other and the 2F5 epitope. For example, the following is a presentation of the HIV-1 gp41 fragment encompassing the 2F5 epitope (shown in bold italic type) and 4E10/Z13 (shown in bold type underlined) epitopes).

Asn Gln Gln Glu Lys Asn Glu Gln Glu Leu Leu Glu

[symbol]

[symbol]

Leu Trp Asn Trp Phe Asn Ile Thr Asn Trp
Cys Cys carrier
i.e. N Q Q E K N E Q E L L E [symbol] L W N W F N I T N W C C-carrier

in one-letter notation.

### EXAMPLE 4

### Presentation of the IgG₁-b12 Epitope

Also, the recently described epitope recognised by the monoclonal antibody IgG₁-b12 which neutralises primary HIV-1 isolates may be similarly modified by insertion of an additional cysteine residue immediately downstream of the reported cysteine residue in order to promote a multimeric presentation by inter-chain disulphide bridging. Addition of residues at the N-terminus leads to further occlusion of the epitope and thus to preferential selection of antibodies with large complementarity-determining regions, and hence of a neutralising phenotype. Both of these modifications to the IgG₁-b12 epitope are shown in the following formula

Ala Glu Gly Glu Phe Ala Ala Ala His [1] Glu Arg Ser Tyr
Met Phe Ser
Asp Leu Glu Asn Arg Cys [14] [symbol] Ile [15]-carrier
i.e. A E G E F A A A A H E R S Y M F S D L E N R C I carrier

in one-letter notation. The IgG₁-b12 epitope sequence (to which the numbering relates; ie His is residue 1, Cys is residue 14 and there is an extra Cys before Ile which is residue 15) is shown in full, with the additional cysteine residue in bold italic type and the additional N-terminal residues in bold type. Cysteine residues are underlined.

### EXAMPLE 5

### Further Presentation of the 2F5 Epitope

An additional example reactive to the 2F5 antibody, based on the consensus sequence of the HIV-1 subtype B is given below:

N-[IAEGEF]-[KNEQELLE]-[LDKWAS]-[LS]-[CC]-[P]-
[DITNWLWYGDPAKAAFDSLQASATEYIGYAWAMVVVIVGATIGI
KLFKKFTSKAS]-C,

where the portions are examples of [A]-[B]-[C]-[D]-[E]-[F]-[G] as described above, in the general description and N and C represent the N and C termini.

This protein was made by PCR amplification of the oligonucleotide Oligo 2 (5′-AAA AAC GAA CAG GAA CTG CTG GAA CTG GAT AAA TGG GCG AGC CTG AGC TGC TGC CCG GTG GAG GGC GCC TGG CGC TGG CGC GG-3′), with primer DC2 (5′-GAT TGA ATT CAA AAA CGA ACA GGA ACT GCT GG-3′) and primer DC5 (5′-AAT TGG ATC CCC GCG CCA GCG CCA GG-3′). 10 pmols of Oligo 2 was used as a template in a 25 ul PCR reaction containing 0.6125 U Hot Start Taq (Qiagen), 100 μM dNTPs (Roche), 200 nM primers DC2 and DC5 in 1×PCR buffer (Qiagen). PCR was carried out as follows: 95° C. for 15 min, then 55 cycles of 94° C. for 45 s, 55° C. for 45 s, 72° C. for 1 min 30 s, with a final extension step of 72° C. for 10 min after which products were held at 4° C. Resulting products were purified using a Qiagen PCR purification kit, then cloned into the pIF4 vector as described previously (Felici et al (1993) Gene 128, 21-27), and the protein expressed from the coding region.

### EXAMPLE 6

### Example of a Protein Sequence that can be Used at [G]

Any protein which has a simple structure (e.g. alpha-helix) could be used as the carrier protein [G] as described in the general description of the invention. For example we have constructed a fusion protein that carries the amino acid sequences described in [A] through to [F] of Example 5 with the minimal colicin R domain at the N terminus (Collins et al (2002) J. Mol. Biol. 318, 787-804), as shown below. This construct was made using fusion (mega-primer) PCR (Miyazaki & Takenouchi (2002) Biotechniques 33, 1033-1038; Brons-Poulsen et al (1998) Molecular & Cellular Probes 12, 345-348; Angelaccio & Bonaccorsi di Patti (2002) Analytical Biochemistry 306, 346-349). Briefly, plasmid containing the colicin R domain (Collins et al (2002) J. Mol. Biol. 318, 787-804) was amplified with primers DC12 (5′-TGG CGC TGG CGC GGG CGA CAG GCT AAA GCT GTT CAG-3′) and CMP10 (5′-CCT CGA GCT CTT TTG CAG CAG C-3′), and the leader sequence and random peptide insert of the 28.3.1 pIF4 plasmid was amplified using primers DC9 (5′-TCA ATG ATG ATG ATG ATG ATG-3′) and DC8 (5′-TGA ACA GCT TTA GCC TGT CGC CCG CGC CAG CGC CAG GC-3′). Resulting products were mixed then re-amplified with primers DC9 and CMP10 in a fusion PCR. Resulting products were then cloned into the pCR3.1.7 TA cloning kit (Invitrogen). Plasmid containing the correct recombinant DNA sequence were exposed to further fusion PCR to add a C-terminal thrombin cleavage site and polyhistadine tail, then cloned into the TOPO Directional pET expression vector (Invitrogen). Recombinant protein expressed in E coli maintains reactivity to 2F5 and is capable of forming oligomers.

N- AEGEFEWEDV EFELDRWALR SCCPVEGAWR WRGRQAKAVQ
VYNSRKSELD AANKTLADAI AEIKQFNRFA HDPMAGGHRM
WQMAGLKAQR AQTDVNNKQA AFDAAAKELE HHHHHH -C

### EXAMPLE 7

### Use of the Fibritin Trimeric Sequence as a Multimerisation Portion

The fibritin timeric sequence (GYIPEAPRDGQAYVRKDGEWVLLSTFL) of the fibritin protein of bacteriophage T4 is used at position [E] to yield a parallel homo-trimeric presentation of the partially occluded 2F5 epitope (Yang et al (2002) J. Virol. 76, 4634-4642; Pakkanen et al (2003) J. Biol. Chem. 278, 32478-32483):

[symbol]

-AEGEF KNEQELLE LDKWAS LS
YIPEAPRDGQAYVRKDGEWVLLSTFL-C

The trimeric region of fibritin has been used to produce stable oligomeric forms of the HIV-1 transmembrane and surface proteins (WO01/19958).

### EXAMPLE 8

### Heterooligomer Formation

Other methods for occluding the epitope might include inserting residues described in [D] through to [F], or those that allow other cross-linking strategies described above, into a different protein (e.g. colicin) with alpha-helical structure, at a position within the protein that would yield partial occlusion of the epitope via the formation of hetero-oligomers with the 2F5-epitope-carrying fusion protein described above.

Thus, one or more of the following polypeptide chains N-RGRQAKAVQV YNSRKSELDL SCCPAANKTL ADAIAEIKQF NRFAHDPMAG GHRMWQMAGL KAQRAQTDVN NKQAAFDAAA KELELVPRGS-C may be oligomerised with one or more of the following polypeptide chain from Example 5: N-[AEGEF]-[KNEQELLE]-[LDKWAS]-[LS]-[CC]-[P]-[DITNWLWYGDPAKAAFDSLQASATEYIGYAWAMVVVIVGATIGIK LFKKFTSKAS]-C, where N and C represent the N- and C-termini.

In all of the trimeric or multimeric presentations of the epitopes described in the Examples and specification above, the conformation may be stabilised by inter-chain disulphide bridging of the reactive peptides or by other chemical means to generate a three-dimensional structure similar to that created by the disulphide-bridged peptides.

The multimeric and/or partially occluded presentations of the reactive peptides according to the invention may be used to induce neutralising antibodies in an immunised host organism (including a suitable cell line). Such presentations may also be used to discriminate between antibodies that neutralise primary HIV-1 isolates, and those that do not. The presentations of the invention and antibodies generated by immunisation with such presentations may be used for the prevention or treatment of HIV-1 infection and AIDS.

1. A molecule which contains a partially occluded and/or multimeric presentation of a peptide which is recognised by an HIV-1 neutralising antibody capable of neutralising diverse clinical isolates of HIV-1.

2. A molecule as claimed in claim 1 wherein the HIV-1 neutralising antibody is any of 2F5, IgG₁-b12, 4E10 and Z13.

3. A molecule as claimed in claim 1 wherein the peptide contained within the molecule is a linear epitope of the HIV-1 neutralising antibody.

4. A molecule as claimed in claim 1 which is a homomultimer of a polypeptide chain which polypeptide chain contains a spacer portion, a linear epitope recognised by the HIV-1 neutralising antibody, a multimerisation portion and, optionally, a carrier portion wherein the polypeptide chain has a molecular weight no more than 30 kDa.

5. A molecule as claimed in claim 1 comprising a portion which is a linear epitope recognised by the HIV-1 neutralising antibody and an occluding portion.

6. A molecule as claimed in claim 1 which is a polypeptide of one or more polypeptide chains.

7. A molecule as claimed in claim 1 which is a homomultimer of a polypeptide chain which contains a linear epitope which recognises the HIV-1 neutralising antibody and an occluding portion wherein the linear epitope is partially occluded by the occluding portion when the polypeptide chain is present in the multimer.

8. A molecule as claimed in claim 1 comprising a first polypeptide chain which contains a linear epitope of the HIV-1 neutralising antibody and a second polypeptide chain which partially occludes the linear epitope on the first polypeptide chain.

9. A molecule as claimed in claim 7 wherein the polypeptide chain contains an occluding portion, the linear epitope, a multimerisation portion and, optionally, a carrier portion.

10. A molecule as claimed in claim 8 wherein the first polypeptide chain contains (1) the linear epitope, (2) a multimerisation portion and, optionally, (3) a carrier portion, and the second polypeptide chain comprises an occluding portion, a multimerisation portion and, optionally, a carrier portion.

11. A trimeric presentation of a peptide as defined in claim 1.

12. A multimeric presentation of a peptide as defined in claim 1 which is stabilised by inter-chain disulphide bridging of the reactive peptides or by other chemical means to generate a three dimensional structure similar to that created by the disulphide-bridged peptides.

13. A polynucleotide encoding a polypeptide chain as claimed in claim 6.

14. (canceled)

15. A pharmaceutical composition comprising a molecule as claimed in claim 1, or a trimeric or multimeric presentation of a peptide as defined in claim 1, or a polynucleotide encoding a peptide as defined in claim 1 and a pharmaceutically acceptable carrier.

16. (canceled)

17. A method of obtaining an HIV-1 neutralising antibody, the method comprising administering a molecule as claimed in claim 1, or a trimeric or multimeric presentation of a peptide as defined in claim 1, or a polynucleotide encoding a peptide as defined in claim 1 to an animal, allowing the animal to produce antibodies, and recovering antibodies directly or indirectly from the animal.

18. A method as claimed in claim 17 wherein the antibodies are monoclonal antibodies.

19. A method of obtaining an HIV-1 neutralising antibody, the method comprising selecting an antibody from an antibody display library in vitro which binds to a molecule as claimed in claim 1, or a trimeric or multimeric presentation of a peptide as defined in claim 1, and synthesising an antibody containing the binding determinants of the so selected antibody.

20. An antibody obtained in accordance with claim 17 or 19 capable of neutralising diverse clinical isolates of HIV-1.

21. (canceled)

22. A pharmaceutical composition comprising an antibody as claimed in claim 20 and a pharmaceutically acceptable carrier.

23. A vaccine for the prevention or treatment of HIV-1 infection which comprises a molecule as claimed in claim 1, or a trimeric or multimeric presentation of a peptide as defined in claim 1, or a polynucleotide encoding a peptide as defined in claim 1.

24. (canceled)

25. A method of treating or preventing HIV-1 infection in an individual the method comprising administering to the individual a molecule as claimed in claim 1, or a trimeric or multimeric presentation of a peptide as defined in claim 1, or a polynucleotide encoding a peptide as defined in claim 1.

26. A method of detecting HIV-1 neutralising antibodies in a sample the method comprising contacting the sample with a molecule as claimed in claim 1 or a trimeric or multimeric presentation of a peptide as defined in claim 1 and determining whether any antibodies present in the sample bind thereto.

27. A method of identifying a molecule which may be useful in raising a neutralising response to HIV-1 the method comprising screening a peptide display library wherein the displayed peptides are from 15 to 40 amino acids in length with an HIV-1 neutralising antibody and selecting those displayed peptides which bind to the antibody.

28. A method according to claim 27 further comprising determining whether the displayed peptides are able to bind to an antibody raised against a linear epitope recognised by the HIV-1 neutralising antibody and selecting those displayed peptides that are not able to so bind.

29. A molecule obtained by the method of claim 27.

30. A method of treating or preventing HIV-1 infection in an individual the method comprising administering to the individual an antibody as claimed in claim 20.
